(19) United States
(12) Patent Application Publication
(10) Pub. No.: US 2024/0130727 A1
(43) Pub. Date: Apr. 25, 2024

(54) SOFT TISSUE REPAIR DEVICE AND METHOD

(71) Applicant: Biomet Sports Medicine, LLC, Warsaw, IN (US)

(72) Inventors: Kevin T. Stone, Winona Lake, IN (US); Nathan M. Sautter, North Manchester, IN (US); Brian K. Berelsman, Warsaw, IN (US); Jeffery D. Arnett, Gilbert, AZ (US); Joshua A. Butters, Chandler, AZ (US); Dylan M. Hushka, Chandler, AZ (US); Nicholas R. Slater, Chandler, AZ (US)

(21) Appl. No.: 18/531,396

(22) Filed: Dec. 6, 2023

Related U.S. Application Data
(63) Continuation of application No. 17/392,971, filed on Aug. 3, 2021.
Application No. 17/392,971 is a continuation of application No. 16/255,300, filed on Jan. 23, 2019, now Pat. No. 11,109,857.
Application No. 16/255,300 is a continuation of application No. 15/332,590, filed on Oct. 24, 2016, now Pat. No. 10,265,064.
Application No. 15/332,590 is a continuation of application No. 13/645,964, filed on Oct. 5, 2012, now Pat. No. 9,504,460.
Application No. 13/645,964 is a division of application No. 12/570,854, filed on Sep. 30, 2009, now Pat. No. 8,303,604.

(51) Int. Cl. A61B 17/06; A61B 17/04

(52) U.S. Cl. CPC A61B 17/06166; A61B 17/0401; A61B 17/0469; A61B 2017/0406; A61B 2017/0409; A61B 2017/0464; A61B 2090/062

(57) ABSTRACT

A soft tissue repair device can include a housing having a handle, a deployment system having an actuation member, and an insertion system having an inserter and a slider. The slider can be coupled to the actuation member and movable relative to the inserter between deployed and retracted positions. First and second anchors can be carried on an external surface of the slider such that the anchors are spaced apart and portions of the anchors are coaxial with the slider and each other. A flexible strand can couple the anchors. The insertion system can cooperate with the deployment system to move the slider to the deployed position to deploy the first anchor upon activating the actuation member a first time, and to move the slider to the deployed position from the retracted position to deploy the second anchor upon actuating the actuation member a second time after the first time.

### CROSS-REFERENCE TO RELATED APPLICATIONS

This application is a divisional of U.S. patent application Ser. No. 12/570,854 filed Sep. 30, 2009, which is a continuation-in-part application of U.S. patent application Ser. No. 12/489,181 filed Jun. 22, 2009, which is a continuation-in-part application of U.S. patent application Ser. No. 12/474,802 filed on May 29, 2009, now U.S. Pat. No. 8,088,130 issued on Jan. 3, 2012, which is a continuation-in-part application of: (1.) U.S. patent application Ser. No. 11/541,506 filed on Sep. 29, 2006, now U.S. Pat. No. 7,601,165 issued on Oct. 13, 2009, and (2.) U.S. patent application Ser. No. 11/541,505 filed on Sep. 29, 2006, now U.S. Pat. No. 7,658,751 issued on Feb. 9, 2010.

This application is a divisional of U.S. patent application Ser. No. 13/045,691 filed on Mar. 11, 2011, which is a continuation-in-part application of U.S. patent application Ser. No. 12/014,399 filed on Jan. 15, 2008, now U.S. Pat. No. 7,909,851 issued on Mar. 22, 2011, which claims the benefit of: (1.) U.S. Provisional Application No. 60/885,062, filed on Jan. 16, 2007, and (2.) U.S. Provisional Application No. 60/885,057, filed on Jan. 16, 2007.

This application is a divisional of U.S. patent application Ser. No. 13/045,689 filed on Mar. 11, 2011, which is a continuation-in-part application of U.S. patent application Ser. No. 12/014,340 filed on Jan. 15, 2008, now U.S. Pat. No. 7,905,904 issued on Mar. 15, 2011, which is a continuation-in-part application of: (1.) U.S. patent application Ser. No. 11/935,681 filed on Nov. 6, 2007, now U.S. Pat. No. 7,905,903 issued on Mar. 15, 2011, and (2.) U.S. patent application Ser. No. 11/869,440 filed on Oct. 9, 2007, now U.S. Pat. No. 7,857,830 issued on Dec. 28, 2010, which is a continuation-in-part of: (1.) U.S. patent application Ser. No. 11/408,282 filed on Apr. 20, 2006 and is now abandoned, (2.) U.S. patent application Ser. No. 11/784,821 filed on Apr. 10, 2007, (3.) U.S. patent application Ser. No. 11/347,661 filed on Feb. 3, 2006 now U.S. Pat. No. 7,749,250 issued on Jul. 6, 2010, (4.) U.S. patent application Ser. No. 11/347,662 filed on Feb. 3, 2006 and is now abandoned, which is a continuation-in-part of U.S. patent application Ser. No. 10/983,236 filed on Nov. 5, 2004 and is now abandoned.

This application is also a continuation-in-part of U.S. patent application Ser. No. 12/489,168 filed Jun. 22, 2009, which is a continuation-in-part of: (1.) U.S. patent application Ser. No. 12/196,405 filed on Aug. 22, 2008, now U.S. Pat. No. 8,128,658 issued on Mar. 5, 2009, (2.) U.S. patent application Ser. No. 12/196,407 filed on Aug. 22, 2008, now U.S. Pat. No. 8,137,382 issued on Mar. 20, 2012, and (3.) U.S. patent application Ser. No. 12/196,410, filed on Aug. 22, 2008, now U.S. Pat. No. 8,118,836 issued on Feb. 21, 2012.

The disclosures of all of the above applications are hereby incorporated by reference.

### FIELD

The present teachings relate generally to soft tissue repair and, more particularly, to a device and associated method for repairing a tear in soft tissue.

### INTRODUCTION

Tears caused by trauma or disease in soft tissue, such as cartilage, ligament, or muscle, can be repaired by suturing. Various repair devices have been developed for facilitating suturing and are effective for their intended purposes. Nevertheless, tissue repair devices for facilitating suturing are still desirable.

### SUMMARY

This section provides a general summary of the disclosure, and is not a comprehensive disclosure of its full scope or all of its features.

According to one aspect, the present teachings provide a soft tissue repair device. The device can include a housing having a handle, a deployment system having an actuation member, and an insertion system having an inserter and a slider. The slider can be coupled to the actuation member and movable relative to the inserter between deployed and retracted positions. At least a portion of a first anchor can be carried on an external surface of the slider in a first position, and at least a portion of a second anchor can be carried on the external surface of the slider in a second position that is longitudinally spaced apart from the first anchor position such that the portions of the first and second anchors are co-axial with the slider and each other. A flexible strand can couple the first and second anchors. The insertion system can be operable to cooperate with the deployment system to move the slider from the retracted position to the deployed position to deploy the first anchor upon actuating the actuation member at a first time, and to move the slider from the retracted position to the deployed position to deploy the second anchor upon actuating the actuation member a second time after the first time.

According to another aspect, the present teachings provide a method for repairing a tear in soft tissue. The method can include coupling first and second flexible anchors with a flexible strand, providing an insertion device having a housing with a pistol grip handle, an inserter and a slider carried in the inserter such that the inserter and the slider each have a distal end extending from the housing. The first and second coupled anchors can be loaded on an external surface of the slider, and the inserter can be inserted though the soft tissue from a first side of the tear to a second side of the tear. An actuation member protruding from the pistol grip handle can be actuated at a first time to translate a distal end of the slider beyond a distal end of the inserter so as to deploy the first anchor. The actuation member can be released after deploying the first anchor, the inserter can be inserted through the soft tissue for a second time in a second position, and the actuation member can be actuated at a second time after the first time to extend the distal end of the slider beyond the distal end of the inserter and deploy the second anchor. The method can further include removing the inserter from the soft tissue, tensioning the flexible strand and reducing the tear.

Further areas of applicability will become apparent from the description provided herein. The description and specific examples in this summary are intended for purposes of illustration only and are not intended to limit the scope of the present disclosure.

### BRIEF DESCRIPTION OF THE DRAWINGS

The present teachings will become more fully understood from the detailed description, the appended claims and the following drawings. The drawings are for illustrative purposes only and are not intended to limit the scope of the present disclosure.

FIG. 1 is a side view of an exemplary tissue repair device according to the present teachings;

FIG. 2 is a top view of the exemplary tissue repair device according to the present teachings;

FIG. 3 is a partial side view of an exemplary configuration of the tissue repair device shown with a portion of the casing removed according to the present teachings;

FIG. 4 is a side view of a portion of the casing of the device of FIG. 1 according to the present teachings;

FIG. 5 is a side view of another portion of the casing of the device of FIG. 1 according to the present teachings;

FIG. 6 is a partial side view of the device of FIG. 1, shown in an exemplary configuration according to the present teachings;

FIG. 7 is a partial side view of the device of FIG. 1, shown in another exemplary configuration according to the present teachings;

FIG. 8 is a partial side view of the device of FIG. 1, shown in another exemplary configuration according to the present teachings;

FIGS. 9A and 9B are perspective views of a cam member of the device of FIG. 1 according to the present teachings;

FIG. 10 is a partial sectional view of the device of FIG. 1, shown in an exemplary configuration according to the present teachings;

FIG. 11 is an exploded perspective view of an exemplary carrying wire, positioning member, insertion member and outer cannula of the device of FIG. 1 according to the present teachings;

FIG. 11A is a partial exploded perspective view of the carrying wire of FIG. 11 having a pair of pre-loaded anchors according to the present teachings;

FIG. 11B is a partial exploded perspective view of the insertion member of FIG. 11 according to the present teachings;

FIG. 11C is a partial exploded perspective view of the outer cannula of FIG. 11 according to the present teachings;

FIG. 12 is a perspective view of the components of FIG. 11, shown in an exemplary assembled configuration according to the present teachings;

FIGS. 13, 14, 15 and 17 are sequential views illustrating an exemplary method and arrangement for coupling first and second flexible anchors with a flexible strand, and FIG. 16 shows a detail of FIG. 15 according to the present teachings;

FIG. 18 is a perspective view of a flexible anchor coupled with a flexible strand according to the present teachings;

FIG. 19 is a partial side view of the device of FIG. 1, shown in an exemplary configuration according to the present teachings;

FIG. 20 is a partial side view of the device of FIG. 1, shown in another exemplary configuration according to the present teachings;

FIG. 21 is an exemplary environmental view showing a first anchor being deployed outside of soft tissue according to the present teachings;

FIG. 22 is an exemplary environmental view showing first and second anchors being deployed outside of soft tissue according to the present teachings; and

FIG. 23 is another exemplary environmental view showing first and second anchors being deployed outside of soft tissue according to the present teachings.

### DESCRIPTION OF VARIOUS ASPECTS

The following description is merely exemplary in nature and is in no way intended to limit the present disclosure, its application, or uses. For example, although the present teachings are illustrated in an application for meniscus repair in knee surgery, the present teachings can also be used for repairing any fibrous tissue, such as muscle, ligament or tendon in an arthroscopic or other open procedure, including rotator cuff reconstruction, acromioclavicular (AC) reconstruction, anterior cruciate ligament reconstruction (ACL) and generally for fastening tendons, grafts, or strands to fibrous tissue and bone.

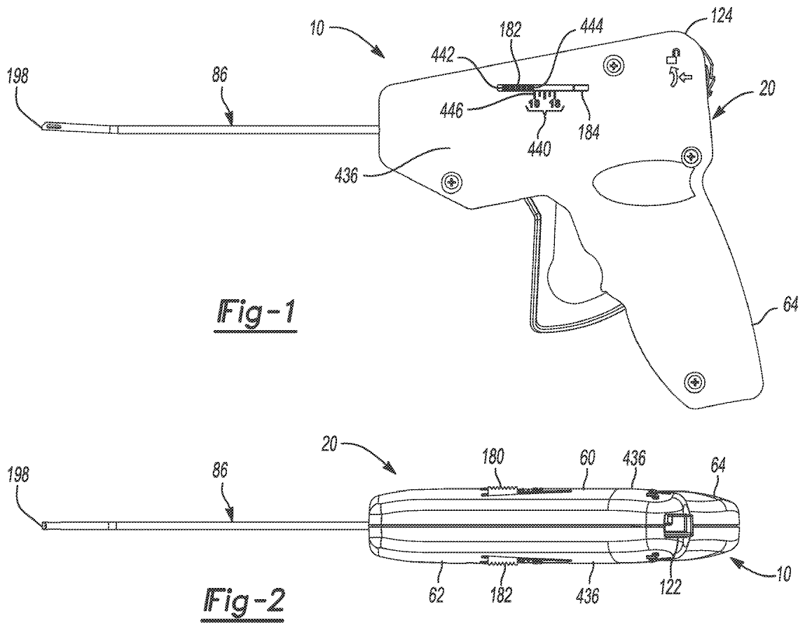

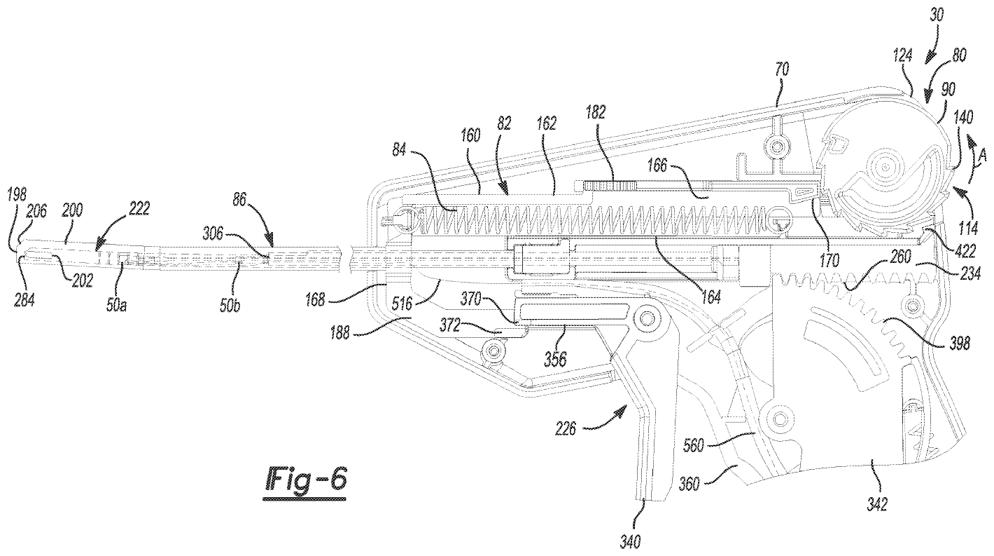

With initial reference to FIGS. 1-6, an exemplary tissue repair device 10 according to the present teachings is illustrated. As will be further described below, the device 10 can include a casing 20 housing a depth limiting or positioning system 30 that cooperates with an insertion system 40, which can be preloaded with one or more flexible suture anchors 50 configured to be delivered into soft tissue. Two flexible suture anchors 50 are illustrated in various Figures, including FIG. 6, and will be generally referred to hereinafter as first and second flexible anchors 50a, 50b for clarification purposes. The casing 20 can include a two-part casing having a first half portion 60 arranged to engage a second half portion 62, such that together the half portions 60, 62 can form a pistol grip handle portion 64. Each casing portion 60, 62 can include a proximal end 66, a distal end 68, a top 70 and a bottom 72 generally located at a distal end of pistol grip handle portion 64. Casing 20 can be formed from plastic or any other suitable material.

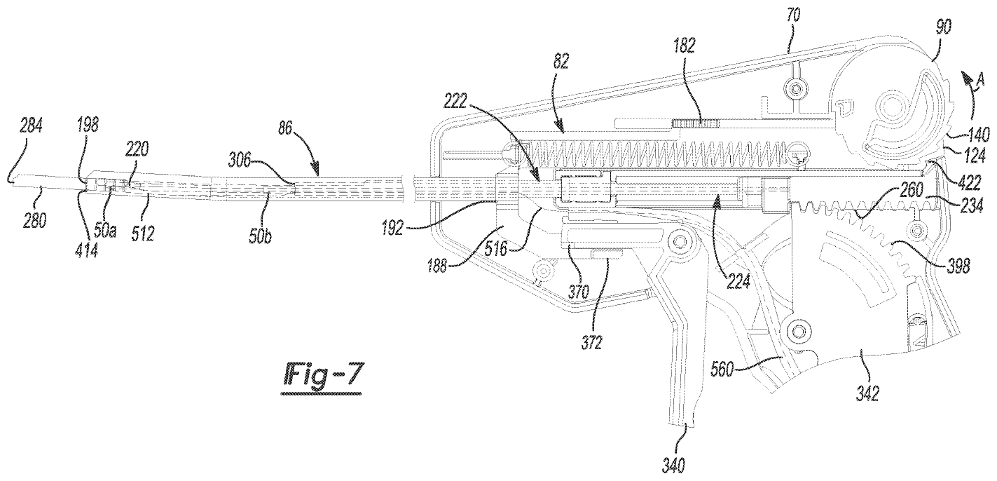

The depth positioning system 30 can include a rotatable cam member 80, an axially moving translation member 82, a biasing member 84, and an axially moveable outer cannula 86. With continuing reference to FIGS. 1-6 and additional reference to FIGS. 7-9B, cam member 80 can include a thumbwheel 90 having a first side 92, a second side 94 and an outer periphery 96. The first side 92 can include a central first projection or post 98 extending from a center thereof that can be positioned in a corresponding slot 100 in first casing portion 60. In a similar manner, second side 94 can likewise include a central second projection or post 104 extending therefrom in an opposite direction as post 98 and can be positioned in a corresponding slot 108 in second casing portion 62. Slots 100, 108 can have a longitudinal length greater than a diameter of respective posts 98, 104 so as to provide for both translation and rotation of thumbwheel 90, as will be described in greater detail below.

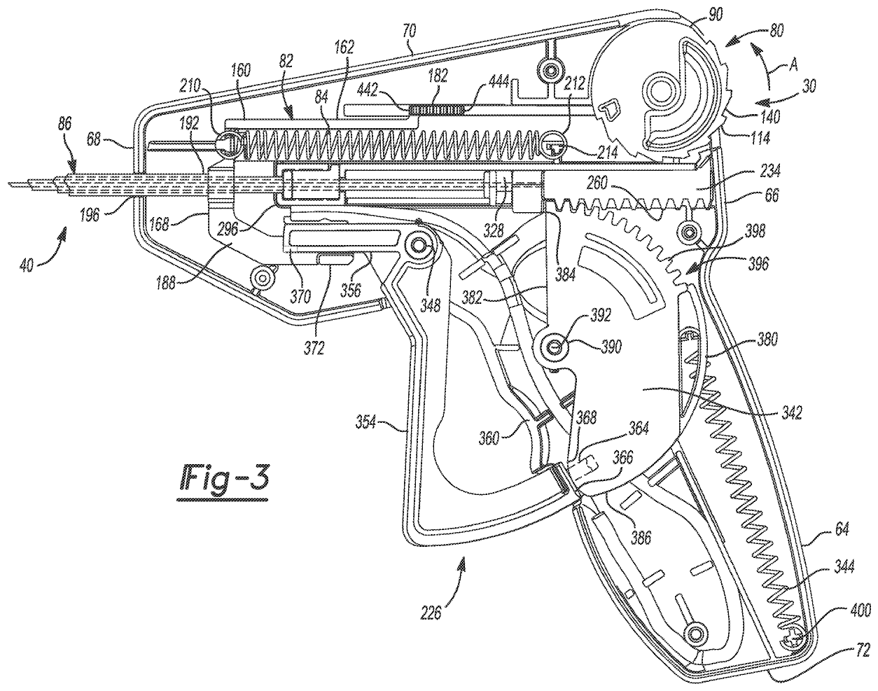

Thumbwheel 90 can further include a toothed portion 114 disposed around a portion of outer periphery 96 and configured to be engagable by a user of the device 10. Casing portions 60, 62 can each include respective recesses 118, 120 that together form an opening 122 to facilitate a portion of thumbwheel 90 extending through the opening 122 and beyond a periphery 124 of casing 20, as shown for example in FIGS. 1 and 3. First side 92 can further include a first rotation limiting surface 130, a first cam surface 132 positioned adjacent thereto, and second and third rotation limiting surfaces 134 and 136, respectively, the operation of which will be described in greater detail below. A recess area 138 can be formed in outer periphery 96 so as to provide access to surfaces 130-136.

The toothed portion 114 can include a plurality of teeth 140 with each tooth having an inclined or ramped surface 142 that mates with second surface 144 orientated generally normal to the outer periphery 96 of thumbwheel 90. The ramped surfaces 142 can be orientated such that they incline away from the outer periphery in a counter-clockwise direction shown by arrow A in FIG. 3.

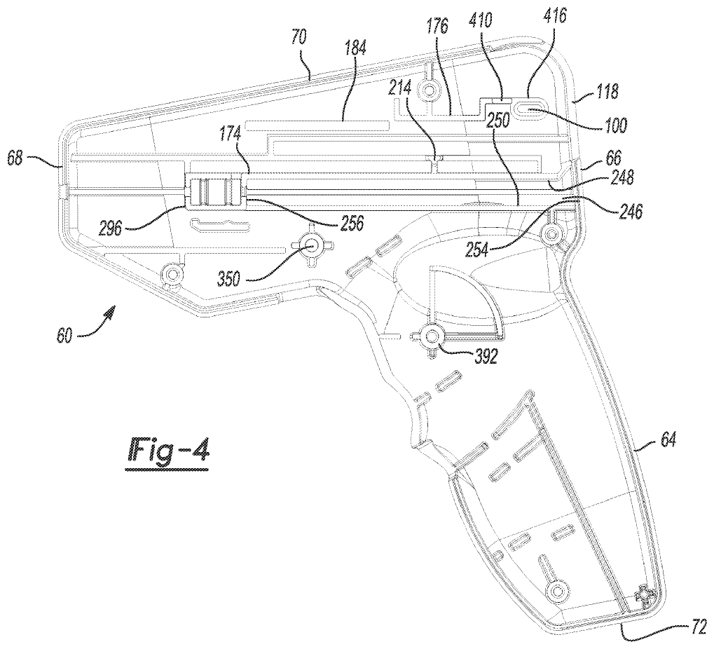

Translation member 82 can include a longitudinally extending portion 160 having a top 162, a bottom 164, a side portion 166 connecting the top and bottom 162, 164, a distal end 168 and a proximal end 170, as shown for example in FIG. 6. Translation member 82 can be positioned in casing 20 such that bottom 164 is adjacent a flange portion 174 of first casing portion 60 and top 162 is adjacent a flange portion 176 also of first casing portion 60, as generally shown in FIG. 4 with reference to FIG. 6. Flange portions 174, 176 can form a channel that guides translation member 82 during translation. A pair of position indication members 180, 182 can extend transversely in opposite directions from top 162 so as to extend through slots 184 in respective casing portions 60, 62 as shown in FIGS. 1 and 2.

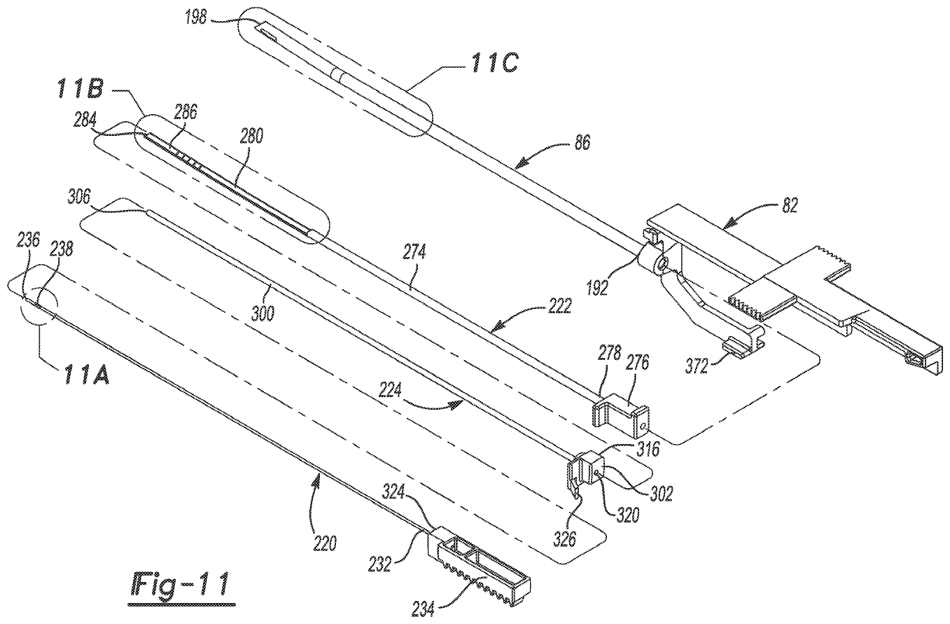

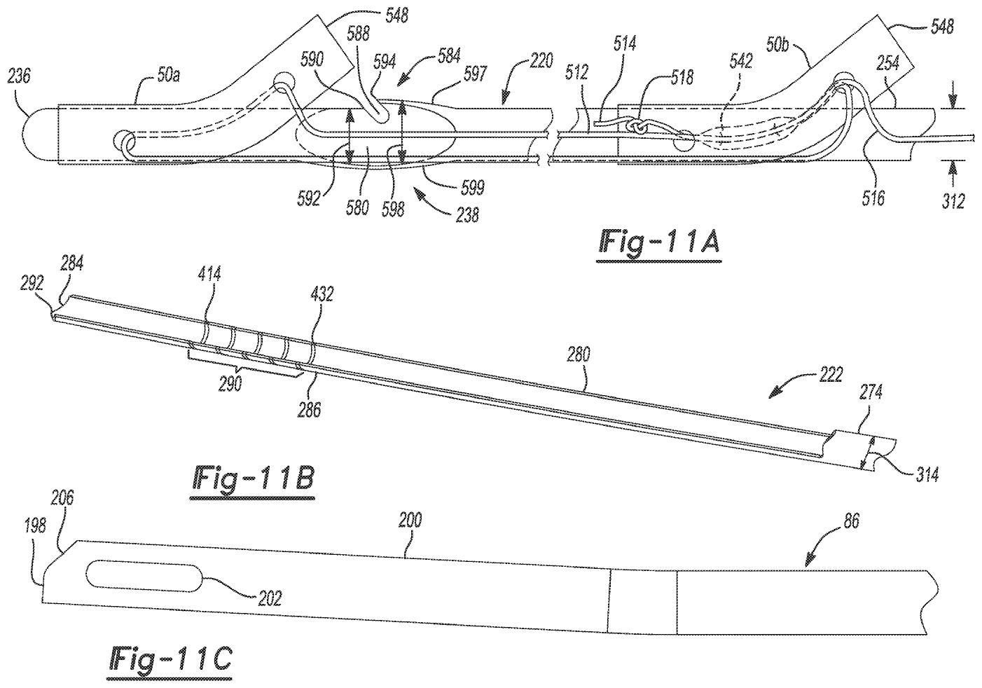

Translation member 82 can further include an L-shaped portion 188 extending from distal end 168, as shown for example in FIG. 6. A proximal end 192 of outer cannula 86 can be coupled to L-shaped portion 188 such that outer cannula 86 can slidably protrude through an aperture 196 formed by casing portions 60, 62. A distal end 198 of outer cannula 86 can include a curved or inclined portion 200 as well as a pair of elongated apertures 202 positioned in alignment on opposite sides of cannula 86, as shown for example in FIG. 11C. Outer cannula 86 can also include a beveled or chamfered end 206 at a distal most end thereof adjacent to apertures 202. Chamfered end 206 can include a rounded or smooth end for placement adjacent to or abutting tissue without piercing the tissue. It should be appreciated that distal end 198 of outer cannula 86 can also be straight or non-inclined, arcuate, angled, etc. depending on a desired use or application, and can also be provided with or without apertures 202 and chamfered end 206.

Biasing member 84 can be connected at a first end 210 to the distal end 168 of translation member 82 and at a second end 212 to a tab 214 fixed to first casing portion 60. Biasing member 84 can be any suitable device, such as a coil spring, configured for expanding and contracting while also exerting a biasing force that biases the proximal end 170 of translation member 82 into engagement with thumbwheel 90.

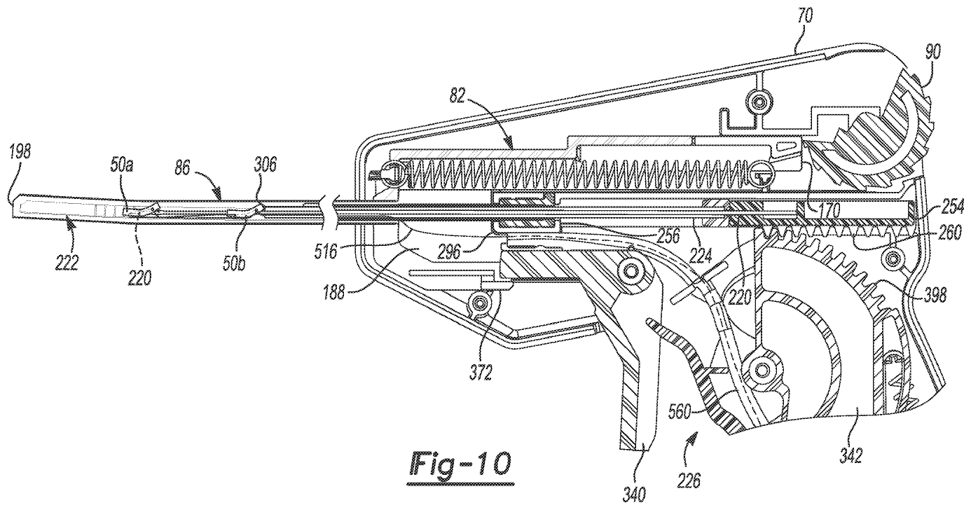

With additional reference to FIGS. 10-12, the insertion system 40 will now be described. Insertion system 40 can include a slider or carrying wire 220, an insertion member 222, a loading or positioning member 224, and a deployment system 226. Carrying wire 220 can include a solid, semi-rigid construction 230 with a proximal end 232 fixedly coupled to a cam follower 234 and a distal end 236 having a suture anchor engagement portion 238. Cam follower 234 can include a generally rectangular shape 242 that can be configured to translate in a track 246 defined by flanges 248 and 250 in first casing portion 60. Track 246 can further include proximal and distal ends 254 and 256 serving to limit travel of the cam follower 234 therebetween, as shown in FIGS. 4 and 10. A lower surface of cam follower 234 can include a plurality of gear teeth 260 configured for meshing engagement with a cam member 342 to translate carrying wire 220 between a stowed position where cam follower 234 abuts proximal end 254 and a deployed position where cam follower 234 abuts distal end 256, as will be further described below.

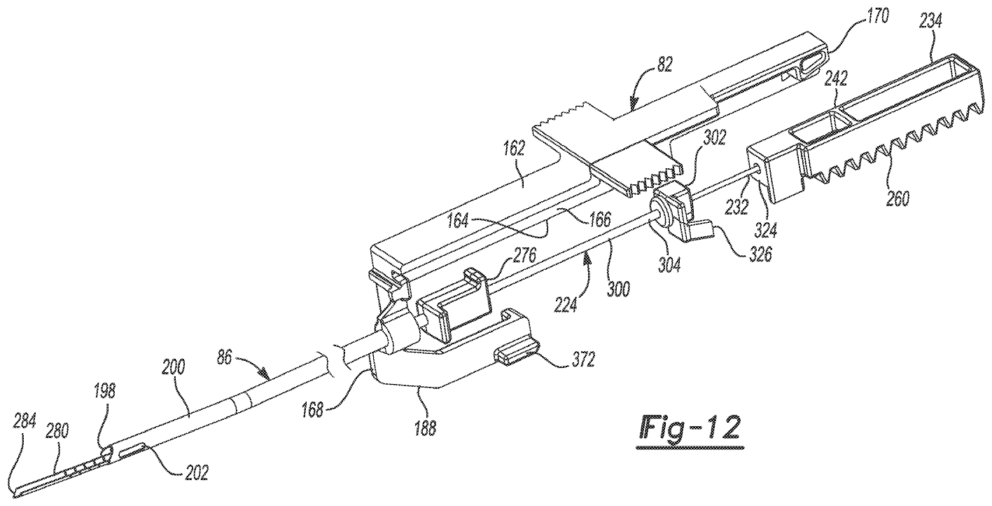

The insertion member 222 can include a tubular portion 274 extending from a body member 276 at a proximal end 278 and a trough portion 280 mating with the tubular portion 274 and extending therefrom to a distal end 284. The trough portion 280 can include an inclined portion 286 complimentary to the inclined portion 200 of cannula 86. It should be appreciated that insertion member 222 can be provided with a straight or non-inclined distal end that could correspond to a straight distal end of outer cannula 86. Inclined portion 286 can include depth indicator markers 290, as well as a pointed and chamfered distal most end 292 capable of piercing skin and soft tissue. Body member 276 can be configured for positioning in a correspondingly shaped cavity 296 to fix insertion member 222 relative to casing 20 and outer cannula 86. The distal end 284 of insertion member 222 can be received in outer cannula 86 such that at least a portion of trough portion 280 and tubular portion 274 can reside within cannula 86 as shown for example in FIGS. 11 and 12.

The positioning member 224 can include a tubular portion 300 fixedly coupled to an engagement member 302 at a proximal end 304, and a distal end 306 configured to engage suture anchor 50b, as will be described in greater detail below in connection with the operation of device 10. Tubular portion 300 can include an inside diameter larger than the suture engagement portion 238 of carrying wire 220 but an outside diameter less than an inside diameter 314 of insertion member 222. Engagement member 302 can include a generally square or rectangular shape 316 configured to mate with the dimensions of track 246 such that engagement member 302 can be slidably received within track 246. Tubular portion 300 can be slidably received within the tubular portion 274 of insertion member 222 such that distal end 306 and a portion of tubular portion 300 are slidably housed within insertion member 222, as shown in FIGS. 11 and 12.

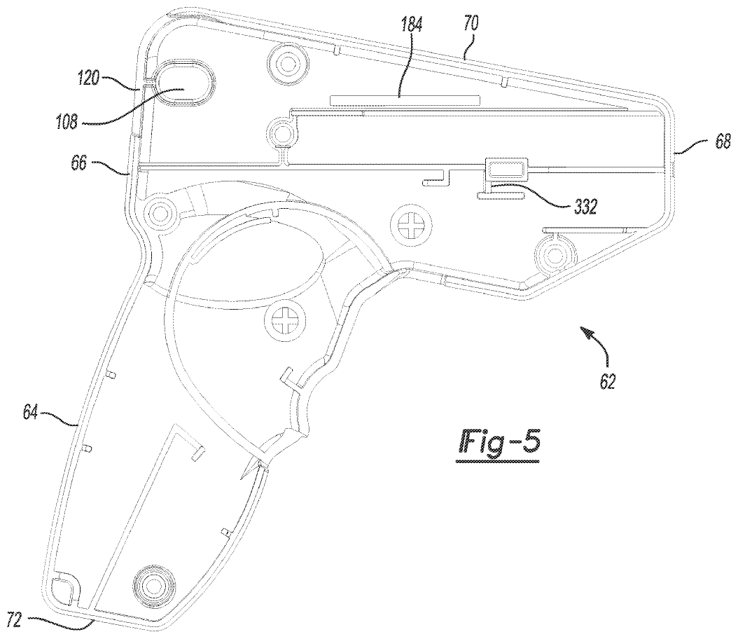

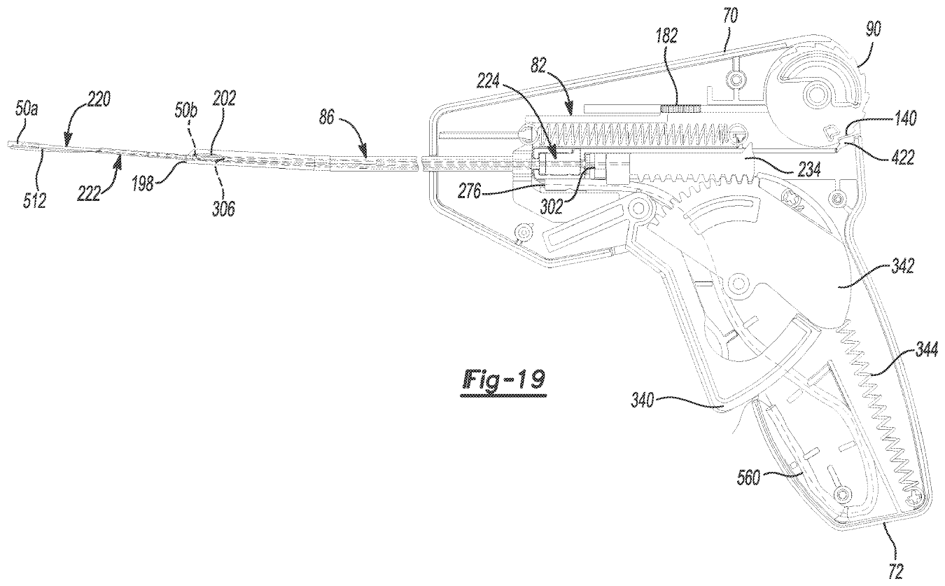

Carrying wire 220 can be inserted through positioning member 224 via aperture 320 such that positioning member 224 can translate on and relative to carrying wire 220. With carrying wire 220 disposed through positioning member 224, engagement member 302 and cam follower 234 can be positioned in track 246 such that engagement member 302 abuts a distal end 324 of cam follower 234 when cam follower 234 is in the stowed position. A retention tab 326 can also extend from a top portion 328 of engagement member 302. Retention tab 326 can be configured to engage a flange 332 extending from casing portion 62 so as to retain engagement member 302 relative to flange 332 when cam follower 234 is translated to the deployed position, as shown in FIG. 19 with reference to FIGS. 5 and 12.

Deployment system 226 can include a trigger 340, a cam member 342 and a biasing member 344. Trigger 340 can include an aperture 348 for pivotally coupling trigger 340 to a pivot post 350 of first casing portion 60, a user engageable portion 354 and a flange member 356. User engageable portion 354 can protrude through an aperture 360 formed by respective casing portions 60, 62 so as to be accessible by a user, as will be further described below. A coupling member 364 can extend from an end 366 of trigger 340 that is configured to abut cam member 342 so as to engage and couple trigger 340 to an end 368 of cam member 342. Flange member 356 can extend from aperture 348 in a direction substantially orthogonal to user engageable portion 354. Flange member 356 can include a tab 370 protruding from a distal end thereof and configured to engage a corresponding tab 372 protruding from L-shaped portion 188, as shown for example in FIG. 6. When tabs 370 and 372 align, trigger 340 is locked such that it can not be rotated from the non-deployed position shown in FIG. 6.

Cam member 342 can include a generally semicircular shaped portion 380 and a generally planer portion 382 spanning between respective ends 384, 386 of portion 380. An aperture 390 can be centrally positioned between ends 384, 386 and configured to engage a pivot post 392 protruding from first casing portion 60 such that cam member 342 can pivot about post 392. Arcuate portion 380 can include a portion 396 having a plurality of gear teeth 398 adjacent to and configured for meshing engagement with the plurality of gear teeth 260 of cam follower 234. Biasing member 344 can be coupled at one end to a retention tab proximate arcuate portion 380 and at another end to a retention post 400 protruding from first casing portion 60. Biasing member 344 can include a coil spring or other device suitable for imparting a biasing force onto cam member 342 to bias trigger 340 to the non-deployed position, as shown for example in FIG. 6.

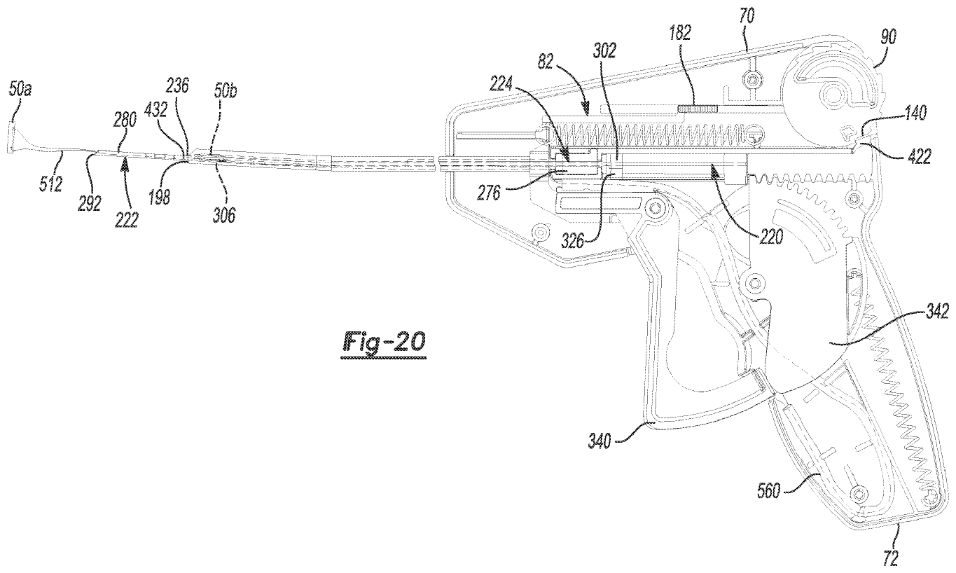

User engageable portion 354 of trigger 340 can be grasped by a user and squeezed or depressed so as to rotate trigger 340 about pivot post 350. Upon such rotation, trigger end 366 can drive cam member 342 to rotate about pivot post 392, which in turn will drivingly engage cam member gear teeth 398 with cam follower gear teeth 260 and translate cam follower 234 and carrying wire 220 forward from the stowed position to the deployed position, as generally shown in FIG. 19. Upon the user releasing trigger 340, the biasing force exerted by biasing member 344 can return cam follower 234, cam member 342 and trigger 340 to the non-deployed positions, as generally shown in FIGS. 6 and 20.

Returning to FIGS. 6-9B, operation of the depth positioning system 30 in connection with the insertion system 40 and trigger lock feature will now be described in greater detail. Thumbwheel 90 can be used to adjust a position of the outer cannula 86 relative to casing 20 and insertion member 222. More specifically, outer cannula 86 can be axially adjusted from a sheathed position to several use positions such that insertion member 222 extends at predetermined lengths relative to distal end 198 of outer cannula 86.

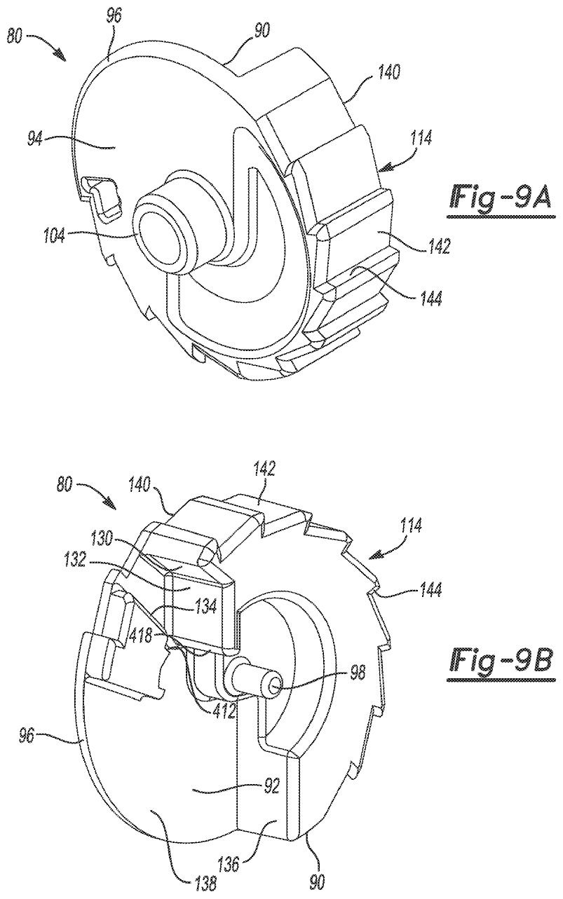

In the sheathed position, thumbwheel 90 can be positioned such that an engagement tab 410 protruding from first casing portion 60 is positioned between a projecting member 412 of rotation limiting surface 134 and an edge 418 of cam surface 132, as generally shown in FIG. 9B with reference to FIG. 4. In this position, thumbwheel 90 can be prevented from rotating and can engage the distal end of translation member 82 at the outer periphery 96 thereby positioning outer cannula 86 to a fully deployed or sheathed position, as shown in FIG. 6. In the sheathed position, outer cannula 86 can fully cover insertion member 222, carrying wire 220 and positioning member 224, as also shown in FIG. 6.

To move the depth positioning system 30 from the sheathed position to an initial use position, a user can push thumbwheel 90 to linearly translate thumbwheel 90 forward towards distal end 68 such that posts 98, 104 slide and translate forward in their respective slots 100, 108 and projection member 412 is disengaged from tab 410. Upon disengagement, thumbwheel 90 can be rotated counter-clockwise in the direction of arrow A such that recessed area 138 is now aligned with the proximal end 170 of translation member 82, as shown in FIG. 7. Biasing member 84 can then urge translation member 82 rearward into recess area 138 such that proximal end 170 engages first cam surface 132, as generally shown in FIG. 7 with additional reference to FIG. 9B. Cam surface 132 can be configured with a shape as shown that cooperates with proximal end 170 of translation member 82 and biasing member 84 to urge thumbwheel 90 to rotate in the direction of arrow A from the sheathed position to the initial use position. In the initial use position, the distal end 198 of outer cannula 86 can align with a first marker 414 of depth markers 290, and a tooth of the plurality of teeth 140 can engage a cooperating projection 422 of first casing portion 60, as also shown in FIG. 7.

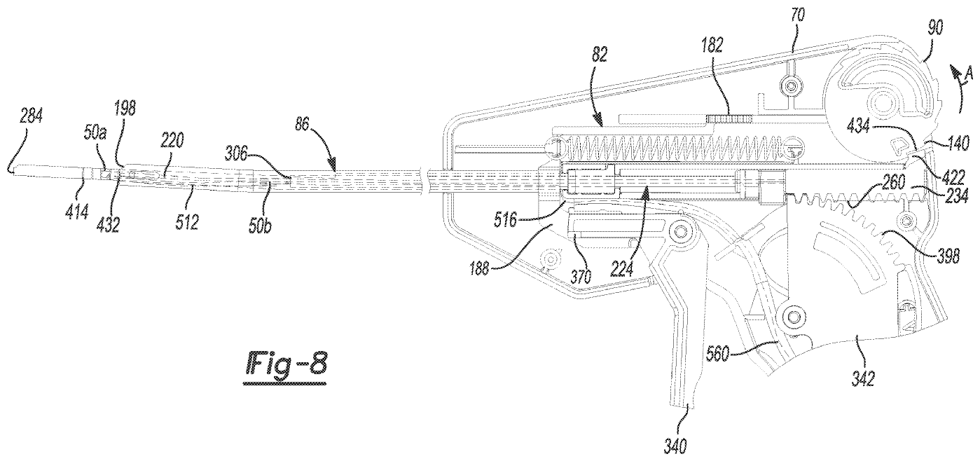

In the initial use position, outer cannula 86 can be partially translated rearward into casing 20 such that a distal end 284 of insertion member 222 is exposed. A user can now rotate thumbwheel 90 to adjust outer cannula 86 to a desired position relative to distal end 284 of insertion member 222. For example, a user could adjust outer cannula 86 relative to distal end 284 such that distal end 284 protrudes a predetermined distance that corresponds to a desired distance in which insertion member 222 can be inserted into the anatomy. In one exemplary configuration, thumbwheel 90 can be used to adjust outer cannula 86 relative to insertion member 222 by 8 millimeters in 2 millimeter increments from first marker 414 to fifth marker 432, as generally shown in FIG. 8. It should be appreciated that the depth and positioning system 30 can be configured to provide various amounts of adjustment as may be desired for various procedures. In this regard, thumbwheel 90 can include a plurality of discrete positions and/or infinite adjustment configurations as may be desired for various procedures.

With continuing reference to FIGS. 6-9B and additional reference to FIGS. 1 and 11B, depth markers 290 can be used to aid a user in positioning outer cannula 86 relative to insertion member 222. In an exemplary configuration, markers 290 and can include five discrete markers or indicia, with the first marker 414 corresponding to a distance of 10 millimeters from the distal end 284 of insertion member 222 to the distal end 198 of outer cannula 86. Each successive marker in a rearward direction towards casing 20 can be separated by 2 millimeters such that the fifth marker 432 can correspond to a distance of 18 millimeters from distal end 284 to distal end 198.

In addition, respective sides 436 of casing 20 can also include depth indicator markings or indicia 440, as shown in FIG. 1. Markings 440 can similarly include five markers that correspond to the same five positions as the markers 290 on insertion member 222. A distal side 442 of position indicator members 180, 182 can be configured to align with markings 440 to provide a visual indication on casing 20 of a position of the distal end 284 of insertion member 222 relative to outer cannula 86, as shown for example in FIG. 1. It should also be noted that when insertion system 40 is in the sheathed position, a proximal side 444 of position indicator members 180, 182 can align with a first marker 446 of markings 440, as shown in FIG. 1.

Each tooth of the plurality of teeth 140 on thumbwheel 90 can be correspondingly spaced apart such that thumbwheel 90 can be used to advance or retract outer cannula 86 in the 2 millimeter increments described above. More specifically, to retract outer cannula 86 away from distal end 284 of insertion member 222, thumbwheel 90 can be pressed forward, such that teeth surfaces 144 clear cooperating projection 422, and then rotated in the counter-clockwise direction of arrow A. For example, thumbwheel 90 can be translated forward and simultaneously rotated in the direction of arrow A to retract outer cannula 86 from the first marker 414 to the fifth marker 432 such that the distal end of insertion member 222 extends beyond outer cannula 86 by a distance of 18 millimeters, as shown in FIG. 8. In this position, second surface 144 adjacent an end of tooth 434 can engage cooperating projection 422 and rotation limiting surface 130 can engage surface 416 of first casing portion 60 adjacent engagement tab 410, as generally shown in FIGS. 8 and 9B. On the other hand, to advance outer cannula 86 towards distal end 284, thumbwheel 90 can be rotated in a direction opposite of arrow A with ramped surfaces 142 ratcheting against cooperating projection 422 to a desired position.

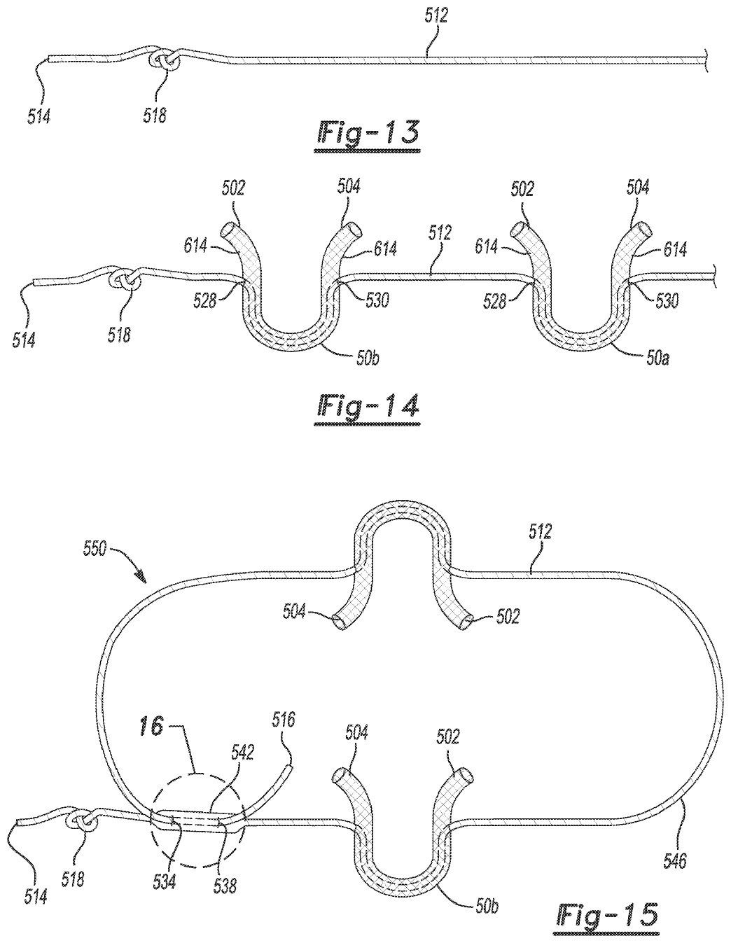

With reference to FIGS. 13-18, the flexible suture anchors 50a, 50b will now be described in greater detail. Each flexible anchor 50a, 50b can be an elongated member having first and second ends 502, 504. The first and second ends 502, 504 can be substantially perpendicular to the longitudinal axis of the flexible anchors 50a, 50b. The flexible anchors 50a, 50b can be made of resorbable or non-resorbable materials, including braided suture, sponges and sponge-like materials in solid form, perforated materials, woven/braided from biocompatible materials or fibers, such as, for example, polymer, polyester, polyethylene, cotton, silk, or other natural or synthetic materials, including sponges and sponge-like materials. The flexible anchors 50a, 50b can have any properties that allow the flexible anchors 50a, 50b to change shape. The flexible anchors 50a, 50b can be, for example, compliant, flexible, foldable, squashable, squeezable, deformable, limp, flaccid, elastic, low-modulus, soft, spongy, perforated or any other flexible member which can change shape. In some aspects, the flexible anchors 50a, 50b can be coated with biological or biocompatible coatings, and can also be soaked in platelets and other biologics, which can be easily absorbed by the flexible anchors 50a, 50b in particular when, for example, the flexible anchors 50a, 50b are made from spongy, absorbent material.

It should be understood by the above description that the flexible anchors 50a, 50b are not configured to pierce or otherwise penetrate tissue either with the first and second ends 502, 504, which are blunt or with any other portion thereof. The flexible anchors 50a, 50b can be loaded on the exterior of the distal end 256 of carrying wire 220, as will be discussed in greater detail below. The flexible anchors 50a, 50b can be in the form of an elongate flexible tube defining a bore 508 along their length, as shown in FIG. 18.

The first and second flexible suture anchors 50a, 50b can be coupled together with a flexible suture or strand 512. The flexible strand 512 can have first and second ends 514, 516 and can be made of braided filaments or fibers of biocompatible material, including natural and synthetic fibers, such as cotton, silk, polymer, polyester, polyethylene, thin wire, suture, and other materials. The flexible strand 512 can be braided in a tubular or hollow form such that it forms an internal passage 520 between the first and second ends 514, 516.

A small knot or other retaining device 518 can be optionally formed adjacent the first end 514. The flexible strand 512 can be passed through a first opening 528 of each of the flexible anchors 50a, 50b, guided along the corresponding bore 508 and passed through a second opening 530 of each flexible anchor 50a, 50b, as for example shown in FIG. 18. The openings 528, 530 can be positioned intermediately between the first and second ends 502, 504 of each flexible anchor 50a, 50b at a distance of, for example, one-quarter length from the ends 502, 504 of each flexible anchor 50a, 50b. Furthermore, it will be appreciated that the openings 528, 530 can be apertures or voids in the woven fabric of the flexible anchors 50a, 50b, such that the openings 528, 530 do not disrupt or break the weave of flexible anchors 50a, 50b, when the flexible anchor 50a. 50b are made of braided or woven material.

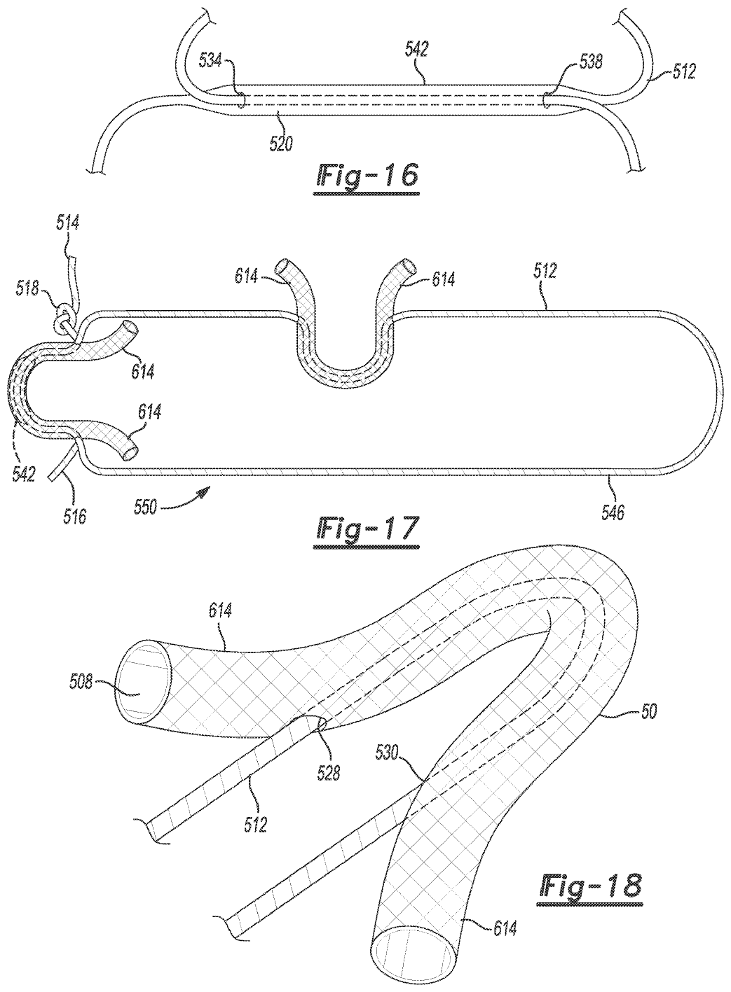

After the flexible anchors 50a, 50b are mounted on the flexible strand 512, the second end 516 of the flexible strand 512 can be inserted into the internal passage 520 of the flexible strand 512 at an aperture 534, guided longitudinally along the passage 520, and led out of the passage 520 of the flexible strand 512 at an aperture 538. The portion of the strand 512 between apertures 534 and 538 can form an adjustment portion 542 between the optional knot 518 and the opening 528 of the second flexible anchor 50b, such that the flexible strand 512 defines a single adjustable knotless loop 546, as shown in FIGS. 15 and 16. The second flexible anchor 50b can be slidably moved along the flexible strand 512 until the adjustment portion 542 is within the bore 508 of the second flexible anchor 50b and the knot 518 is adjacent the opening 528 of the second flexible anchor 50b, as shown in FIG. 17. It will be appreciated, however, that the adjustment portion 542 can remain in the position shown in FIG. 15 outside of bore 508. The adjustable knotless loop 546 is self-locking and does not require the surgeon to tie a knot during a surgical procedure for securing the flexible strand 512. Further, once the adjustable knotless loop 546 is self-locked by pulling the second end 516 of the flexible strand 512 and tensioning the flexible strand 512, friction prevents the adjustable knotless loop 546 from being loosened, thereby providing a secure lock. Additional details regarding forming the knotless adjustable loop 546, and additional adjustable knotless loop configurations are disclosed in co-pending and commonly assigned U.S. patent application Ser. No. 11/541,506, filed on Sep. 29, 2006, the disclosure of which is incorporated herein by reference.

For discussion purposes, the anchors 50a, 50b, together with flexible strand 512 configured in the knotless loop 546 can be hereinafter referred to as the anchor system 550. With reference to FIGS. 6 and 11A, the anchor system 550 can be pre-loaded in an in-line configuration on the carrying wire 220 such that anchor 50a is positioned before flexible anchor engagement portion 238 and second anchor 50b is positioned after engagement portion 238. In the in-line configuration, carrying wire 220 can extend through a portion of bore 508 of anchors 50a, 50b and then pierce though each anchor thereby creating a tail portion 548 that is not coaxial with carrying wire 220, as shown for example in FIG. 11A. In this configuration, the anchors 50a, 50b can reside in the trough portion 280 of insertion member 222. The flexible strand 512 can be packaged within outer cannula 86 along side insertion member 222 and positioning member 224. Second end 516 of flexible strand 512 can extend out of outer cannula 86 through aperture 196 and into a tubular housing member 560 positioned in a handle portion of first casing portion 60, as shown for example in FIG. 6.

Returning to FIGS. 11 and 11A, the suture anchor engagement portion 238 of carrying wire 220 will now be described in greater detail. Suture engagement portion can have a flattened area 580 having a thickness less than the outside diameter 312 of carrying wire 220. Flattened area 580 can be formed so as to maintain the same cross-sectional area as a cross section through a non-flattened portion of carrying wire 220, thereby providing for the suture anchors 50a, 50b to be sized relative to the diameter of carrying wire 220 while ensuring that they can slide over the engagement portion 238, as will be further described below.

The engagement portion 238 can further include a one-way barb 584 configured such that flexible anchors 50a, 50b can slide or travel over barb 584 in only one direction of travel from the proximal end 254 of carrying wire 220 to the distal end 236 thereof. One-way barb 584 can be defined by a recess or cut-out 588 formed in the flattened section such that a distal end 590 of an open end of recess 588 mates with a portion of flattened area 580 having a first width 592. A proximal end 594 of the recess 588 also adjacent the opening can mate with another portion of the flattened area 580 having a second width 598 greater than the first width 592. Flattened area 580 can also include curved transition areas 597, 599 between the flattened area 580 and a non-flattened area of the carrying wire on a proximal side thereof, as shown in FIG. 11A.

In use, the larger second width 598 of flattened area 580 and the angled nature of one-way barb 584 provides for suture anchor 50b to be able to slide or travel over the barb 584 in a proximal to distal direction without being caught or stopped by barb 584. In a similar manner, carrying wire 220 can slide or travel relative to suture anchor 50b in a distal to proximal direction without barb 584 catching or stopping flexible anchor 50b. Conversely, the smaller first width 592 of flattened area 580 in cooperation with barb 584 provides for catching an interior surface of bore 508 of suture anchor 50b when carrying wire 220 moves in a proximal to distal direction relative to flexible anchor 50b.

Referring now to FIGS. 6-8 and 19-23, the soft tissue repair device 10 pre-loaded with suture anchors 50a, 50b can be used to repair a soft tissue defect 600, such as, for example, a tear, or other weakness in fibrous soft tissue 602, such as in meniscal tissue, cartilage, muscle or other fibrous tissue under the skin. An outer incision 601 can be made through the skin to access the soft tissue 602 and a user can insert the distal end 198 of outer cannula 86 to the surgical site. An appropriate insertion depth can be determined for the insertion member 222 and the user can translate and rotate thumbwheel 90, as discussed above, to unlock the depth and positioning system 30 from the sheathed position to the initial use position. The outer cannula 86 can then be adjusted relative to insertion member 222 such that distal end 284 extends beyond outer cannula 86 a distance corresponding to the desired insertion depth.

The exposed distal end 284 of insertion member 222 can be inserted through first entry point 604 into soft tissue 602 from a first side of the defect 600 until the distal end 284 can exit a second side 606 of the fibrous soft tissue 602, such as an outer surface or back side of a meniscus of a knee joint or other outer surface of a fibrous tissue. In this position, the distal end 198 of outer cannula 86 can be adjacent to or abutting the first entry point 604. It should be appreciated that the chamfered end 292 of insertion member 222 could alternatively be used to pierce the skin without requiring an incision 604.

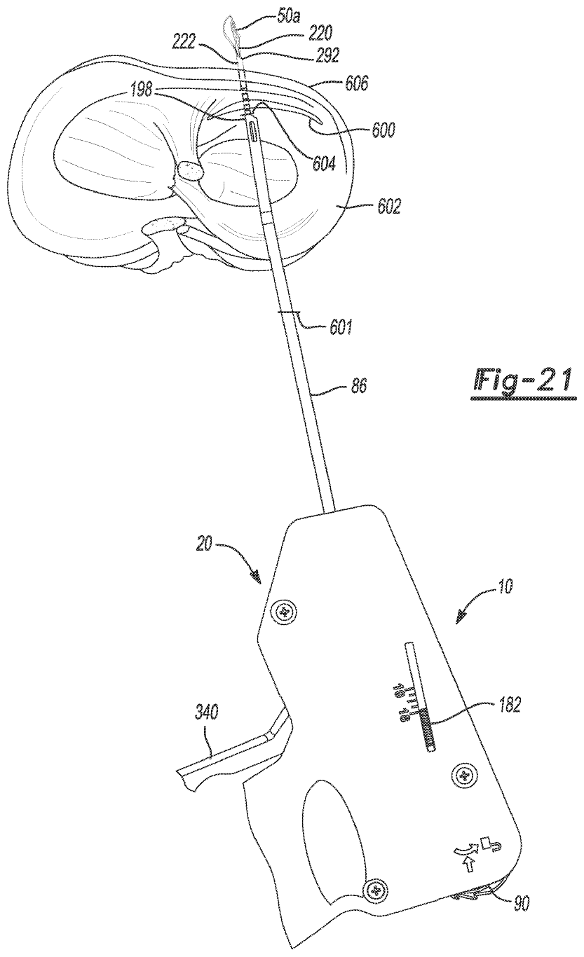

With distal end 284 of insertion member 222 appropriately positioned, the trigger 340 can be squeezed or depressed to translate carrying wire 220 forward relative to insertion member 222 such that one-way barb 584 engages first flexible suture anchor 50a and delivers anchor 50a on the second side 606 of the soft tissue 602 at a first location, as shown in FIGS. 19 and 21. It should be appreciated that the manner and structure of the pre-assembled anchor system 550 in-line on carrying wire 220 allows the anchor 50a to pass through a narrow opening or slit, first entry point 604, formed in the tissue 602 by the chamfered end 292 of insertion member 222. It should also be appreciated that the U-shaped trough portion 280 of insertion member 222 requires a smaller insertion area than a fully enclosed, circular shaped insertion member would require and will not core a plug of tissue.

As cam follower 234 translates carrying wire 220 forward to deploy first anchor 50a in connection with depressing trigger 340, cam follower 234 also simultaneously translates positioning member 224 forward such that distal end 306 engages second flexible anchor 50b and moves anchor 50b forward towards distal end 198 of outer cannula 86, as shown in FIGS. 19 and 20. Tubular portion 300 of positioning member 224 can have a length such that when trigger 340 is fully depressed to deploy first anchor 50a, second anchor 50b will be carried forward to a position adjacent apertures 196 and distal end 198 of outer cannula 86. In this position, retention tab 326 can engage flange 332 to retain positioning member 224 and thus anchor 50b in this advanced position when trigger 340 is released. After the first anchor 50a has been deployed, the trigger 340 can be released thereby retracting the distal end 284 of insertion member 222 such that one-way barb 584 is positioned rearward of or within bore 508 of suture anchor 50b, as generally shown in FIGS. 19 and 21. When insertion member 222 is retracting, the tail portion 548 of suture anchor 50a can engage the soft tissue side 606 such that it will be removed from insertion member 222 as insertion member 220 retracts to the stowed position.

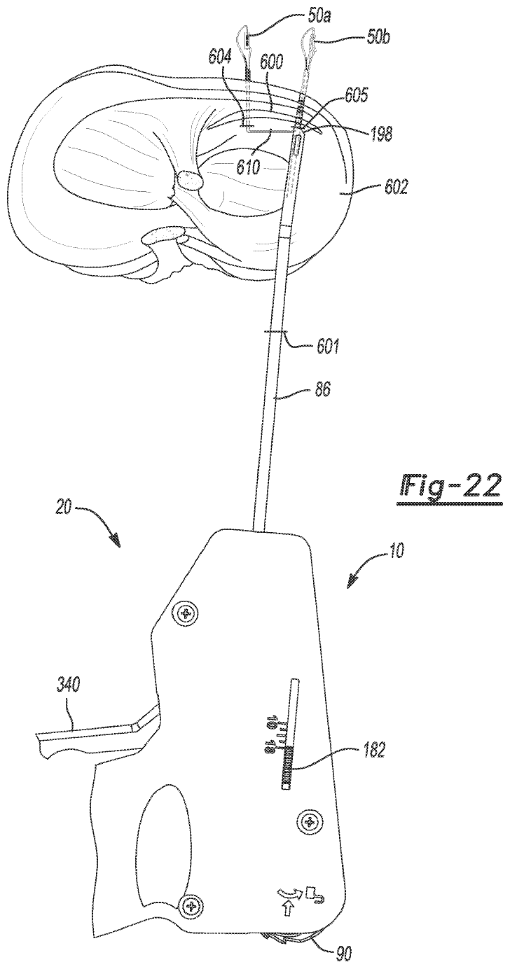

With the first anchor 50a deployed, the insertion device 10 can be removed from the soft tissue 602 while portions 610 of the knotless loop 546 can slide out from insertion device 10 as flexible anchor is retained in soft tissue 602. Insertion device 10 can then be inserted at a second location or entry point 605 on a second side of the soft tissue defect 600, as shown in FIG. 22. Once distal end 284 of insertion member 222 is appropriately positioned, such as beyond second side 606, trigger 340 can again be squeezed or depressed to deploy second anchor 50b adjacent to first anchor 50a. Depressing trigger 340 again translates carrying wire 220 forward such that one-way barb 584 engages an interior of bore 508 and deploys second flexible anchor 50b at the second location such that tail portion 548 of anchor 50b can engage soft tissue side 606 upon retraction on insertion member 222, as also generally shown in FIG. 22. Insertion device 10 can then be removed from the soft tissue 602 and the skin incision 601 with the second end 516 of the flexible strand 512 being removed from the soft tissue 602 along with insertion device 10, as also shown in FIG. 22. Once second anchor 50b is implanted and insertion device 10 removed from the soft tissue 602, any remaining portion of second flexible strand end 516 not removed from outer cannula 86 and housing member 560 can be subsequently removed.

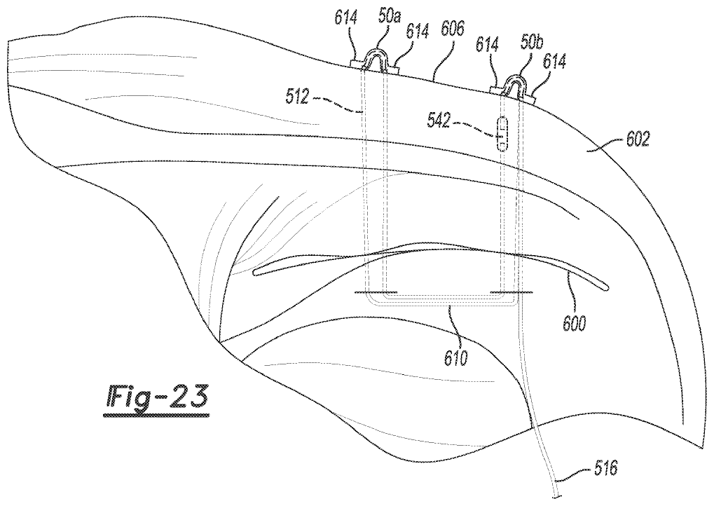

Pulling the second, free end 516 of the flexible strand 512 can tighten the adjustable knotless loop 546, secure the first and second flexible anchors 50a. 50b against the second side surface 606 of the soft tissue 602, and reduce the defect 600. Further, portions 614 of the anchors 50a, 50b between the first and second ends 502, 504 and the corresponding first and second openings 528, 530, can define anchoring leg portions 614 that can provide additional resistance for securing the flexible anchors 50a, 50b on the surface 606 of the soft tissue 602, as these leg portions can be forced against surface 606 for anchoring, as shown for example in FIG. 23.

It will be appreciated from the above description and drawings that the present teachings provide flexible anchors that can be passed through tissue easily in a compact or low profile configuration and or orientation and then positioned outside tissue in a second orientation that provides anchoring without tissue penetration, preventing withdrawal from the tissue and reducing tissue injury. Further, the use of an inserter provided with preassembled anchors can help reduce the time length of the procedure and simplify manipulations required during the procedure.

It will be further understood that the various aspects of the depth positioning system, insertion system, deployment system and anchors can be mixed and matched or combined in ways other than those explicitly discussed above, without departing from the scope of the present teachings.

The foregoing discussion discloses and describes merely exemplary arrangements of the present disclosure. One skilled in the art will readily recognize from such discussion, and from the accompanying drawings and claims, that various changes, modifications and variations can be made therein without departing from the spirit and scope of the present teachings as defined in the following claims.

1.-20. (canceled)

21. A meniscal tissue repair device, comprising:
a user-graspable casing that includes a first casing portion attached to a second casing portion;
an inserter extending from the casing, the inserter including an open distal end and a longitudinal bore that extends through the inserter to the open distal end;
a first flexible suture anchor deployable from the inserter at a first location along an outer surface of a meniscus;
a second flexible suture anchor deployable from the inserter at a second location along the outer surface of the meniscus, wherein, in a pre-deployment condition of the meniscal tissue repair device, the second flexible suture anchor is located proximal of the first flexible suture anchor along the inserter;
a suture coupling the first flexible suture anchor to the second flexible suture anchor;
a cam pivotable about a pivot point that is located within the casing, the cam including a first plurality of teeth that extend in an arcuate manner along the cam;
a cam follower including a second plurality of teeth that extend in a linear manner along the cam follower, the second plurality of teeth meshing with the first plurality of teeth such that pivoting the cam about the pivot point causes the cam follower to move linearly within the casing;
a user-accessible element protruding from an aperture in the casing so as to be accessible and movable by a user from outside the casing, said aperture formed by attaching the first casing portion to the second casing portion, the user-accessible element coupled to the cam such that movement of the user-accessible element by the user causes the cam to pivot about the pivot point; and
a carrying wire slidably received in the longitudinal bore of the inserter, the carrying wire including a proximal portion coupled to the cam follower such that linear movement of the cam follower within the casing causes the carrying wire to move linearly within the longitudinal bore of the inserter, the carrying wire further including an anchor engagement portion distal of the proximal portion, wherein, in the pre-deployment condition of the meniscal tissue repair device, the carrying wire extends distally past the second flexible suture anchor toward the first flexible suture anchor to position the anchor engagement portion between the second flexible suture anchor and the first flexible suture anchor in the longitudinal bore of the inserter,
wherein, from the pre-deployment condition of the meniscal tissue repair device, the cam is pivotable about the pivot point in a first rotational direction to advance the carrying wire within the longitudinal bore of the inserter and thereby force the first flexible suture anchor from the inserter via an engagement of the first flexible suture anchor with the anchor engagement portion,
wherein the cam is pivotable about the pivot point in a second rotational direction opposite the first rotational direction to retract the carrying wire within the longitudinal bore of the inserter and thereby move the anchor engagement portion proximally past at least part of the second flexible suture anchor, and
wherein the cam is pivotable about the pivot point in the first rotational direction to advance the carrying wire within the longitudinal bore of the inserter and thereby force the second flexible suture anchor from the inserter via an engagement of the second flexible suture anchor with the anchor engagement portion.

22. The meniscal tissue repair device of claim 21, wherein the user-accessible element includes an end portion adjoining the cam.

23. The meniscal tissue repair device of claim 22, wherein the end portion is rotatable about an axis.

24. The meniscal tissue repair device of claim 21, wherein the cam follower has a rectangular shape.

25. The meniscal tissue repair device of claim 21 further comprising an anchor stop, wherein, in the pre-deployment condition of the meniscal tissue repair device, the second flexible suture anchor is located between the first flexible suture anchor and the anchor stop.

26. A meniscal tissue repair device, comprising:
a user-graspable casing;
an inserter extending from the casing, the inserter including an open distal end and a longitudinal bore that extends through the inserter to the open distal end;
a first flexible suture anchor deployable from the inserter at a first location along an outer surface of a meniscus;
a second flexible suture anchor deployable from the inserter at a second location along the outer surface of the meniscus, wherein, in a pre-deployment condition of the meniscal tissue repair device, the second flexible suture anchor is located proximal of the first flexible suture anchor along the inserter;
a suture coupling the first flexible suture anchor to the second flexible suture anchor;
a cam pivotable about a pivot point that is located within the casing, the cam including a first plurality of teeth that extend in an arcuate manner along the cam;
a cam follower including a second plurality of teeth that extend in a linear manner along the cam follower, the second plurality of teeth meshing with the first plurality of teeth such that pivoting the cam about the pivot point causes the cam follower to move linearly within the casing; and
a carrying wire slidably received in the longitudinal bore of the inserter, the carrying wire including a proximal portion coupled to the cam follower such that linear movement of the cam follower within the casing causes the carrying wire to move linearly within the longitudinal bore of the inserter, the carrying wire further including an anchor engagement portion distal of the proximal portion, wherein, in the pre-deployment condition of the meniscal tissue repair device, the carrying wire extends distally past the second flexible suture anchor toward the first flexible suture anchor to position the anchor engagement portion between the second flexible suture anchor and the first flexible suture anchor in the longitudinal bore of the inserter,
wherein, from the pre-deployment condition of the meniscal tissue repair device, the cam is pivotable about the pivot point in a first rotational direction to advance the carrying wire within the longitudinal bore of the inserter and thereby force the first flexible suture anchor from the inserter via an engagement of the first flexible suture anchor with the anchor engagement portion,
wherein the cam is pivotable about the pivot point in a second rotational direction opposite the first rotational direction to retract the carrying wire within the longitudinal bore of the inserter and thereby move the anchor engagement portion proximally past at least part of the second flexible suture anchor, and
wherein the cam is pivotable about the pivot point in the first rotational direction to advance the carrying wire within the longitudinal bore of the inserter and thereby force the second flexible suture anchor from the inserter via an engagement of the second flexible suture anchor with the anchor engagement portion.
wherein the carrying wire includes a non-flattened section that is located proximal of the anchor engagement portion, the non-flattened section having a non-flattened diameter and a non-flattened cross-sectional area,
wherein the carrying wire also includes a flattened section that is part of the anchor engagement portion, the flattened section including at least a first flattened portion,
wherein the first flattened portion has: (i) a first width that is greater than the non-flattened diameter of the non-flattened section of the carrying wire; (ii) a first thickness that is less than the non-flattened diameter of the non-flattened section of the carrying wire; and (iii) a first cross-sectional area that is equal to the cross-sectional area of the non-flattened section of the carrying wire.

27. The meniscal tissue repair device of claim 26, wherein the flattened section of the carrying wire also includes a second flattened portion having a second width that is greater than the first width.

28. The meniscal tissue repair device of claim 26, wherein the flattened section includes at least a first curved transition area distal of the non-flattened section of the carrying wire.

29. The meniscal tissue repair device of claim 26, wherein, in the pre-deployment condition of the meniscal tissue repair device, the carrying wire extends through the second flexible suture anchor.

30. The meniscal tissue repair device of claim 26, wherein the engagement of the first flexible suture anchor with the anchor engagement portion includes the anchor engagement portion contacting an intermediate portion of the first flexible suture anchor, the intermediate portion located between a first end and a second end of the first flexible suture anchor.

31. A meniscal tissue repair device, comprising:
a user-graspable casing that includes a first casing portion attached to a second casing portion;
an inserter extending from the casing, the inserter including an open distal end and a longitudinal bore that extends through the inserter to the open distal end;
a first flexible suture anchor deployable from the inserter at a first location along an outer surface of a meniscus;
a second flexible suture anchor deployable from the inserter at a second location along the outer surface of the meniscus, wherein, in a pre-deployment condition of the meniscal tissue repair device, the second flexible suture anchor is located proximal of the first flexible suture anchor along the inserter;
a suture coupling the first flexible suture anchor to the second flexible suture anchor;
a cam pivotable about a pivot point that is located within the casing, the cam including a first plurality of teeth that extend in an arcuate manner along the cam;
a cam follower including a second plurality of teeth that extend in a linear manner along the cam follower, the second plurality of teeth meshing with the first plurality of teeth such that pivoting the cam about the pivot point causes the cam follower to move linearly within the casing;
a user-accessible element protruding from an aperture in the casing so as to be accessible and movable by a user from outside the casing, the user-accessible element coupled to the cam such that movement of the user-accessible element by the user causes the cam to pivot about the pivot point; and
a carrying wire slidably received in the longitudinal bore of the inserter, the carrying wire including a proximal portion coupled to the cam follower such that linear movement of the cam follower within the casing causes the carrying wire to move linearly within the longitudinal bore of the inserter, the carrying wire further including an anchor engagement portion distal of the proximal portion, wherein, in the pre-deployment condition of the meniscal tissue repair device, the carrying wire extends distally past the second flexible suture anchor toward the first flexible suture anchor to position the anchor engagement portion between the second flexible suture anchor and the first flexible suture anchor in the longitudinal bore of the inserter,
wherein the cam is pivotable about the pivot point: (i) a first time and in a first rotational direction to advance the carrying wire distally within the longitudinal bore of the inserter and thereby force the first flexible suture anchor from the inserter via an engagement of the first flexible suture anchor with the anchor engagement portion for deploying the first flexible suture anchor from the inserter at the first location along the outer surface of the meniscus; (ii) a second time and in a second rotational direction opposite the first rotational direction to retract the carrying wire proximally within the longitudinal bore of the inserter and thereby move the anchor engagement portion proximally past at least part of the second flexible suture anchor; and (iii) a third time and back in the first rotational direction to advance the carrying wire distally within the longitudinal bore of the inserter and thereby force the second flexible suture anchor from the inserter via an engagement of the second flexible suture anchor with the anchor engagement portion for deploying the second flexible suture anchor from the inserter at the second location along the outer surface of the meniscus,
wherein the carrying wire includes a non-flattened section that is located proximal of the anchor engagement portion,
wherein the carrying wire also includes a flattened section that is part of the anchor engagement portion.

32. The meniscal tissue repair device of claim 31, wherein the non-flattened section of the carrying wire has a non-flattened diameter and a non-flattened cross-sectional area.

33. The meniscal tissue repair device of claim 32, wherein the flattened section of the carrying wire includes at least a first flattened portion.

34. The meniscal tissue repair device of claim 33, wherein the first flattened portion has a first width that is greater than the non-flattened diameter of the non-flattened section of the carrying wire.

35. The meniscal tissue repair device of claim 34, wherein the flattened section includes at least a first curved transition area distal of the non-flattened section of the carrying wire.

36. The meniscal tissue repair device of claim 33, wherein the first flattened portion has a first thickness that is less than the non-flattened diameter of the non-flattened section of the carrying wire.

37. The meniscal tissue repair device of claim 36, wherein the flattened section includes at least a first curved transition area distal of the non-flattened section of the carrying wire.

38. The meniscal tissue repair device of claim 33, wherein the first flattened portion has a first cross-sectional area that is equal to the cross-sectional area of the non-flattened section of the carrying wire.

39. The meniscal tissue repair device of claim 38, wherein the flattened section includes at least a first curved transition area distal of the non-flattened section of the carrying wire.

40. The meniscal tissue repair device of claim 38, wherein the first flattened portion has a first width that is greater than the non-flattened diameter of the non-flattened section of the carrying wire.

41. The meniscal tissue repair device of claim 40, wherein the flattened section of the carrying wire also includes a second flattened portion having a second width that is greater than the first width.

42. The meniscal tissue repair device of claim 31 further comprising an anchor stop, wherein, in the pre-deployment condition of the meniscal tissue repair device, the second flexible suture anchor is located between the first flexible suture anchor and the anchor stop.
